(19) United States
(12) Patent Application Publication
(10) Pub. No.: US 2010/0298760 A1
(43) Pub. Date: Nov. 25, 2010

(54) METHODS, KITS, AND COMPOSITIONS FOR ADMINISTERING PHARMACEUTICAL COMPOUNDS

(76) Inventors: Bernat Olle, Cambridge, MA (US); Daphne Zohar, Boston, MA (US); Jonathan Behr, Boston, MA (US); David Steinberg, Milton, MA (US)

Correspondence Address: CLARK & ELBING LLP, 101 FEDERAL STREET, BOSTON, MA, 02110, US

(21) Appl. No.: 12/741,338

(22) PCT Filed: Oct. 21, 2008
(86) PCT No.: PCT/US08/11979

§ 371 Date: Aug. 9, 2010

Related U.S. Application Data
(60) Provisional application No. 60/985,612, filed on Nov. 5, 2007.

(51) Int. Cl. A61M 5/00; A61N 5/067; A61B 17/34

(52) U.S. Cl. 604/20; 604/22; 604/46

(57) ABSTRACT

The invention features methods, kits, and compositions for administering pharmaceutical compounds using microdermabrasion particles.

### BACKGROUND OF THE INVENTION

The invention relates to methods, kits, and compositions for delivering compounds to a tissue, and more particularly to methods for treating a skin-related condition, comprising disrupting the skin (e.g. by removing one or more layers of the skin) and embedding drugs in the skin.

Some existing methods for skin disruption, such as microdermabrasion, involve removing the most superficial layer of the skin by propulsion of particles or a liquid jet. However, conventional microdermabrasion procedures do not result in significant embedding of particles. Most particles do not have sufficient momentum density (momentum divided by the cross sectional area of the particle) to penetrate past the stratum corneum. Accidental embedding of microdermabrasion particles during procedures is generally considered undesirable, because it can lead to granuloma formation. Consequently, manufacturers of microdermabrasion devices adjust the operating parameters (particle size, particle density, suction pressure, particle velocity, number of passes) so that embedding of particles is minimized.

Typical microdermabrasion procedures use relatively large particles, on the order of 100-150 micron, which are convenient for obtaining substantial skin disruption, but are not necessarily desirable for applications that require embedding of a particle into the skin. embedding of particles in the range of 0.1 to 250 micron is possible, typical particle sizes for drug delivery may be on the order of 10 micron.

Methods for embedding particles in the skin for transdermal drug delivery have been described in the prior art. These techniques, referred to as ‘velocity-based’ or ‘needle-free’, are based on propelling particles at very high speeds (generally supersonic), in order to breach the stratum corneum and penetrate the underlying tissues. Use of compressed helium to accelerate solid drug particles through a Venturi nozzle at velocities of up to 800 m/s has been reported by Bellhouse et al, U.S. Pat. No. 5,630,796. U.S. Pat. No. 7,207,967 describes a velocity-based method for accelerating drug-containing particles with an average size of 10-70 micron at velocities ranging from 200 to 3000 msec. Appropriate pressure to accelerate the particles is obtained by a transient supersonic helium gas jet. U.S. Pat. No. 6,893,664 describes a method that makes use of a needleless syringe whereby the penetration depth of the particles propelled can be controlled by adjusting the extent to which a gas container is breached, which restricts the outflow of gas from the container. Some examples of needle-free devices under development include Intraject®, Implaject®, Jet Syringe®, Iject®, Mini-ject®, Crossjet®, and PowderJect®. For example, the PowderJect® device consists of a gas canister that allows pressurized helium gas to enter a chamber that contains a cassette filled with drug. The powdered drug sits between two polycarbonate membranes, which are instantaneously ruptured when the gas is released; this, in turn, causes the gas to expand rapidly, forming a shock wave that progresses down the nozzle at speeds of 600-900 m/s. The particle velocity is controlled by the nozzle geometry, the burst strength, and the gas pressure.

Existing transdermal technologies based on propelling drug particles against the skin generally have several limitations. First, a fraction of the drug propelled gets retained in the stratum corneum. Using the PowderJect device, Lahm and Lee (J of Pharm Sci, 95, 7, 2006) have shown that the ratio of drug that remains in the stratum corneum to drug that crosses beyond is roughly 10:1. As a result, an important fraction of the drug administered is wasted. A second problem with existing technologies is that particles accelerated at supersonic speeds typically collide strongly against each other resulting in significant particle attrition; by the time such particles collide with the skin, their size distribution has been shifted to a lower value (Lahm and Lee, J of Pharm Sci, 95, 7, 2006). As a result, it is difficult to have precise control of the size of the particles delivered to the skin, which in turn may play an important role in the release pattern of the drug and the depth and retention of embedded particles. Other problems that have been associated with velocity-based techniques are the lack of reliability and occasional bruising.

Other technologies for transdermal delivery of drugs do not involve propelling particles at high velocities, but instead rely on removing or modifying the stratum corneum, which constitutes the main resistance to drug transport through the skin, and then applying a drug topically. These devices rely on techniques widely used by dermatologists, such as dermabrasion, or light-based methods. One such device for enhancing the delivery of topical drug formulations has been developed by Med Pharm Ltd and is described in WO05058226A1. Another technique currently in development by Carlisle Scientific, relies on creating microchannels in the skin by disrupting the stratum corneum with sharp metal granules (microscissuining). In the context of skin abrasion-based techniques for enhancing drug delivery, microdermabrasion devices have been used solely for the purpose of removing the stratum corneum, but not for embedding a drug. Instead, the drug has been applied in a topical formulation following the abrasion step.

Skin abrasion-based techniques for enhancement of topical drug delivery can have several limitations. First, while removing part or all of the stratum corneum typically increases the permeability of the skin to agents, some molecules are too large to penetrate the remaining layers of the skin. For example, large proteins, or drugs that must be formulated into carrier particles (e.g. controlled release depots), may not readily diffuse through the epidermis or dermis. In addition, in existing approaches, where the drug is applied topically, it will inevitably be distributed into the skin through a concentration gradient that develops over time, with the highest concentration being at the skin surface. As a result, high concentrations of drug may be difficult to obtain in deep layers of the skin over relevant time scales (e.g. hours, days, or weeks), or high concentrations of drug may be impossible to avoid in the outer layers (e.g. the stratum corneum or the epidermis), which may not be the desired target of the treatment. Third, in some cases it may be necessary for the skin to remain uncovered after the drug application; a topically applied drug may wash off through friction (e.g. with clothes) or contact with water, while embedded drug particles would not.

### SUMMARY OF THE INVENTION

The invention features methods, kits, and compositions for delivering compounds to a tissue, and more particularly methods for treating a skin-related condition comprising disrupting the skin (e.g. by removing a layer of the skin) and embedding drugs in the skin.

Given the limitations of both velocity-based and skin abrasion-based drug delivery methods, a technique that combined the advantages of the two would be desirable. The methods of the invention include continuous transdermal delivery of particles (a) taking place at lower particle velocities, lower particle sizes, and lower particle densities than those needed in velocity-based devices, while (b) maintaining a high penetration efficiency into the skin by removing the stratum corneum, and (c) retaining good control over the depth and distribution of the drug in the skin. In this invention, the incorporation of smaller particle sizes can be used to effect embedding in the skin without forming granulomas, and for optimizing drug delivery. The compositions of the invention feature microdermabrasion particles containing pharmaceutical compounds formulated for controlled release. In certain cases it may be desirable to improve permeation by propelling solid particles of these drugs against the skin, as opposed to applying them in a topical formulation (e.g. cream, gel, foam). Second, in some cases it may be desirable to insert the drug at a specific depth in the skin so that it lies near a specific structure of the skin (e.g. the epidermis, dermis, the hair bulge, the hair papilla, the sebaceous gland, etc).

In one aspect, the invention features a method of delivering (e.g., using a transdermal delivery device or microdermabrasion device) a pharmaceutical compound (e.g., a therapeutic or cosmetic compound) to a tissue (e.g., an internal or external tissue) including continually propelling particles onto the tissue where at least some of the particles include a therapeutic compound, at least some of the particles (e.g., 0.1%, 1%, 5%, 10%, 20%, 30%, 50% or more) embed in the tissue, and the therapeutic compound is released into the tissue.

In another aspect, the invention features a method of delivering a pharmaceutical compound (e.g., a therapeutic or cosmetic compound) to a tissue including embedding particles into the tissue where at least some of the particles include a therapeutic compound by propelling at least some of the particles (e.g., 0.1%, 1%, 5%, 10%, 20%, 30%, 50% or more) into the tissue; and releasing the therapeutic compound into the tissue.

In another aspect, the invention features a microdermabrasion particle formulated for controlled release of a pharmaceutical compound. The microdermabrasion particle can be formulated to melt at least in part at temperatures, for example, between body temperature and 60° C., melt at least in part at body temperature, or melt between room temperature and body temperature. Such microdermabrasion particle may be a mixture of high melting point fats and low melting point fats. Such microdermabrasion particles may also be formulated to stick to the tissue (e.g., skin). The microdermabrasion particle may have at least one property selected from the group consisting of: a high surface charge or polarity, carboxylic acids, poly(anhydride) groups, high molecular weight polymers, and polymers with high chain flexibility. The diameter of the microdermabrasion particle can be between 0.01 μm to 200 μm (e.g., 0.01 μm, 0.05 μm, 0.1 μl, 0.5 μm, 1 μm, 10 μm, 25 μm, 50 μm, 75 μm, 100 μM, 150 μm, 175 μm, and 200 μm in diameter.

In another aspect, the invention also features a microdermabrasion device including a handpiece, a tip, a propellant, and a cartridge selected from the group consisting of a cartridge containing therapeutic compound particles formulated into an abrasive carrier and a cartridge containing a mixture of abrasive particles and therapeutic compound particles.

In yet another aspect, the invention features a microdermabrasion device including a handpiece, a tip, a propellant, a cartridge containing abrasive particles and a cartridge containing therapeutic compound particles.

In another aspect, the invention features a microdermabrasion device including a handpiece, a tip, a propellant, and a cartridge containing therapeutic compound particles, wherein the therapeutic compound particles are formulated into an abrasive solid carrier.

In yet another aspect, the invention features a microdermabrasion kit for use with a microdermabrasion device, the kit including a cartridge and a tip, wherein the cartridge includes microdermabrasion particles and wherein the microdermabrasion particles include a therapeutic compound formulated for controlled release. This kit may also feature a recycling unit and/or a collection unit.

In any of the forgoing aspects, the particles can be embedded to a depth of between 0.01 mm and 7 mm (e.g., 10-30 μm, 30-100 μm, 500 μm, 800 μm, 2 mm, and 5 mm).

In any of the forgoing aspects, particles can be a mixture of particles containing a therapeutic compound and particles that do not contain a therapeutic compound. In this aspect, particles containing a therapeutic compound may differ in size or shape from those that do not contain a therapeutic compound. In a related aspect, the above method also features the selective removal of the non-therapeutic compounds on the basis of size or shape. Such particles may be collected, recycled, and/or purified on the basis of size or shape. In another related aspect, the invention features the selective collection, recycling, and/or purification of the particles containing the pharmaceutical compound based on, for example, size or shape.

In another related aspect, the particles can be a mixture of particles containing differing pharmaceutical compounds. Mixtures of particles can differ based on size and shape. Such particles can be propelled simultaneously or in sequence to different depths depending on the size and or shape of the differing particles.

In any of the forgoing aspect, methods can further include disrupting the tissue (e.g., using a microdermabrasion device). This disruption can be in an amount to trigger an embryonic like state and/or reepithelialization in the tissue (e.g., skin). In this aspect, the compound can be administered prior to, simultaneous with, or after disruption of the tissue. Also in this aspect, the therapeutic compound can be administered in an amount sufficient to enhance hair follicle neogenesis, inhibit follicle neogenesis or hair growth, prevent or treat an aging related skin condition, treat a pigmentation disorder, treat a growth, or treat acne.

This disruption can result in removal of tissue to a depth of between 0.01 and 7 mm. For example, the disruption can result in removal of at least one skin component selected from the group consisting of the stratum corneum, a portion of the epidermis, the full epidermis, a portion of the dermis, the full dermis, the sebaceous glands, the bulges, and the dermal papillas.

In any of the above methods or compositions, the therapeutic compound can be formulated for controlled release. For example, the compound can be formulated for delayed released (after a period of 1, 2, 3, 4, 5, 6, 7, 8, 9, and 10 days) or sustained released (over a period of 1, 2, 3, 4, 5, 6, 7, 8, 9, and 10 days). The controlled release can be activated by endogenous sources (e.g., temperature, chemicals, pressure, water, cell secretions, enzymes, dissolved gases, and reactive oxygen species) or exogenous sources (e.g., electromagnetic radiation, electric current, light, heat, chemicals, pressure, ultrasound, water, solvents, catalysts, and enzymes).

Also in any of the forgoing methods or compositions, the therapeutic compound can be a small molecule EGFR inhibitor, or metabolite thereof (e.g., a non-naturally occurring nitrogen-containing heterocycle of less than about 2,000 daltons, leflunomide, gefitinib, erlotinib, lapatinib, canertinib, vandetanib, CL-387785, PKI166, pelitinib, HKI-272, and HKI-357), EGF, an EGFR antibody (zalutumumab, cetuximab, IMC 11F8, matuzumab, SC 100, ALT 110, PX 1032, BMS599626, MDX 214, and PX 1041), a suppressor of the expression of a Wnt protein in the hair follicle or an inducer of expression of a Dkk1 protein (e.g., from lithium chloride, a molecule that synergizes with lithium chloride, the agonists 6-bromoindirubin-3_-oxime, deoxycholic acid, a pyrimidine derivative, antagonists quercetin, ICG-001, the purine derivative QS11, fungal derivatives PKF115-854 and CGP049090, and the organic molecule NSC668036), a modulator the retinoic acid signaling pathway (trans-retinoic acid, N-retinoyl-D-glucosamine, and seletinoid G), a modulator of the estrogen signaling pathway (e.g., 17β-estradiol and selective estrogen receptor modulators), a compound which modulates the ubiquitin-proteasome system, a compound which modulates cytokine signaling of Imiquimod or IL-1alpha, a modulator of melanocortin signaling, tyrosinase activity, apoptosis signaling, endothelin signaling, nuclear receptor signaling, TGFβ-SMAD signaling, bone morphogenetic protein signaling, stem cell factor signaling, androgen signaling, retinoic acid signaling, peroxisome proliferator-activated response receptor signaling, estrogen signaling, cytokine signaling, growth factor signaling, nonandrogenic hormone signaling, toll-like receptor signaling, and neurotrophin, neuroendocine signaling, and cytokine signaling, benzoyl peroxide, a photosenitizer (e.g., aminolevulinic acid), an interferon, dacarbazine, interleukin-2, imiquimod, or a promoter of the expression of the transcription factor MITF.

By the terms “embed” and “embedding” are meant fixing or setting securely or deeply, a particle, within or below the surface of the tissue.

By “pharmaceutical compound” is meant any compound that, when contacted with a tissue, results in therapeutic, cosmetic, or prophylactic activity.

The terms “administration” and “administering” refer to a method of giving a dosage of a pharmaceutical composition to a patient, where the method is, e.g., topical, oral, intravenous, transdermal, subcutaneous, intraperitoneal, or intramuscular.

As used herein, “reepithelialization” refers to the process that occurs during formation of a new epidermis after wounding. Tissue undergoing this process may be characterized by the lack of fully developed hair follicles, cells in an embryonic-like state, or by lack of a stratum corneum.

As used herein, to “promote differentiation” refers to the act of increasing the percentage of cells that will differentiate as indicated or to increase the number of cells per unit area of skin that will differentiate.

By “uncommitted epidermal cell” is meant an epidermal stem cell, a bulge cell, a bulge-derived cell, or any other type of cell known in the art that can be induced to differentiate into an HF cell.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteroids may be halogenated. Examples corticosteroids are provided herein.

By “disruption” is meant a sufficient amount of disturbance to existing tissue (e.g., hair follicles and the surrounding epidermis and/or dermis) to induce an “embryonic-like” state. This embryonic-like state includes the activation, migration, and differentiation of epithelial stem cells from the bulge region of the hair follicle or the interfollicular epidermis. The depth of skin disruption can include in increasing amounts: partial removal of the stratum corneum, complete removal of the stratum corneum, partial removal of the epidermis, complete removal of the epidermis, partial disruption of the dermis and complete removal of the dermis. Skin disruption can also include disruption of the mid to lower epidermis and/or dermis without any disturbance to the stratum corneum and/or outer epidermis. Different levels of skin disruption can be accomplished by chemical, energetic, mechanical, sound, ultrasound, and/or electromagnetic based methods.

By “controlled release” is meant the regulated spatial and/or temporal release of a therapeutic compound from a formulation. The term “controlled release” is meant to include delayed release, sustained release, and release from the formulation in pulses or cyclical patterns. The controlled release of the compound may be activated by an exogenous or endogenous stimulus.

By “delayed release” is meant that the therapeutically active component is not immediately released from the formulation (e.g., a carrier particle).

By “sustained release” is meant a form of controlled release whereby the therapeutically active compound is released over an extended period of time.

As used herein, “formulated for topical administration” refers to a composition of the invention containing a therapeutic, cosmetic, or prophylactic compound and formulated with a pharmaceutically acceptable excipient to form a dispersible composition. Compositions formulated for topical administration (e.g., as a cream, gel, lotion, ointment, microdermabrasion particle, and any other topical formulation described herein) are those manufactured or sold in accordance with governmental regulations regarding a therapeutic, prophylactic, or cosmetic regimen that includes instructions for the topical administration of the composition.

By “microdermabrasion” is meant a technique for skin disruption that uses propulsion of particles or a liquid jet. The term is also meant to include a technique for skin disruption that uses a small, reciprocating, hard tip (e.g., a diamond).

By “microdermabrasion particle” is meant a composition, that when propelled onto the skin, results in disruption of the skin. The term “microdermabrasion particle” is meant to include both compositions comprising a therapeutic compound and compositions which themselves have no therapeutically active compounds. Microdermabrasion particles may include frozen solutions containing a therapeutic compound or may include formulations of therapeutic compounds that are solid at room temperature.

By “microdermabrasion device” is meant a device for skin disruption that uses propulsion of particles or a liquid jet. The term is also meant for a device for skin disruption that uses a small, reciprocating, hard tip (e.g., a diamond). As described herein, microdermabrasion devices may propel frozen particles, or particles that are solid at room temperature or at the temperature that the procedure takes place.

By “recycling unit” is meant a device that separates propelled microdermabrasion particles, or the therapeutic compound contained therein, from a fraction of cellular debris and other byproducts of skin disruption resulting from microdermabrasion.

By “collection unit” is meant a device that collects the propelled microdermabrasion particles, cellular debris, and other byproducts of skin disruption resulting from microdermabrasion.

By “small molecule EGFR inhibitor” is meant a molecule that inhibits the function of one or more EGFR family tyrosine kinases. Tyrosine kinases of the EGFR family include EGFR, HER-2, and HER-4 (see Raymond et al., Drugs 60(Suppl. 1):15 (2000); and Harari et al., Oncogene 19:6102 (2000)). Small molecule EGFR inhibitors include, for example, gefitinib (Baselga et al., Drugs 60(Suppl. 1):33 (2000)), erlotinib (Pollack et al., J. Pharm. Exp. Ther. 291:739 (1999)), lapatinib (Lackey et al., 92^{nd} AACR Meeting, New Orleans, abstract 4582 (2001)), canertinib (Bridges et al., Curr. Med. Chem. 6:825 (1999)), vandetanib (Wedge et al., Cancer Res. 62:4645 (2002)), CL-387785 (Discafani et al., Biochem. Pharmacol. 57:917 (1999)), PKI166 (Takada et al., Drug Metab. Dispos. 32:1272 (2004)), pelitinib (Torrance et al., Nature Medicine 6:1024 (2000)), HKI-272, HKI-357 (for HKI-272 and HKI-357 see, for example, Greenberger et al., 11^{th} NCI-EORTC-AACR Symposium on New Drugs in Cancer Therapy, Amsterdam, abstract 388 (2000); Rabindran et al., Cancer Res. 64:3958 (2004); Holbro et al., Ann. Rev. Pharm. Tox. 44:195 (2004); Tsou et al., J. Med. Chem. 48:1107 (2005); and Tejpar et al., J. Clin. Oncol. ASCO Annual Meeting Proc. 22:3579 (2004)), and leflunomide (Kochhar et al., FEBS Lett. 334:161 (1993)). The structures for each of these compounds is provided below in Table 1.

TABLE 1
EGFR Inhibitors

| Drug | Structure |
|---|---|
| leflunomide | 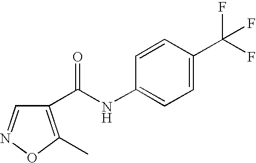 |
| Grefitinib | 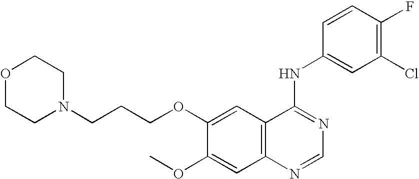 |
| Erlotinib | 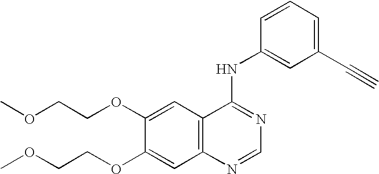 |
| Lapatinib | 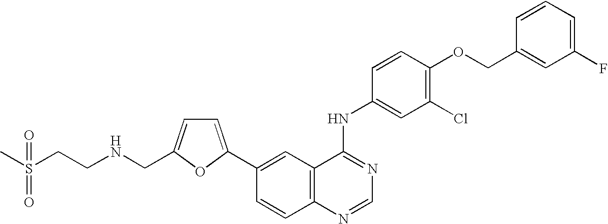 |
| Canertinib | 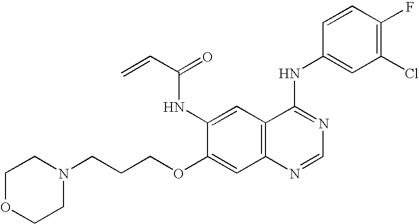 |
| Vandetanib | 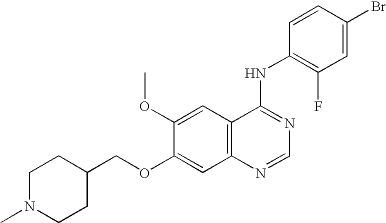 |
| CL-387785 | 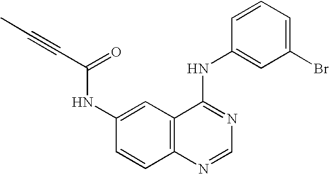 |
| PKI166 | 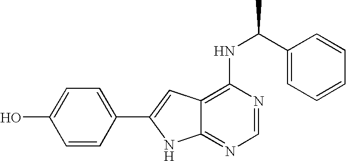 |
| Pelitinib | 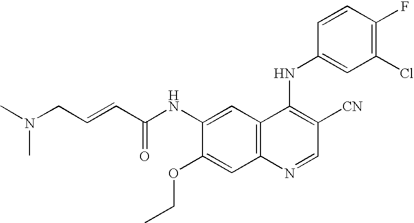 |
| HKI-272 | 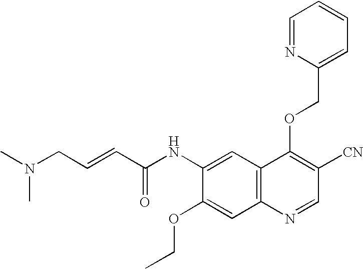 |
| HKI-357 | 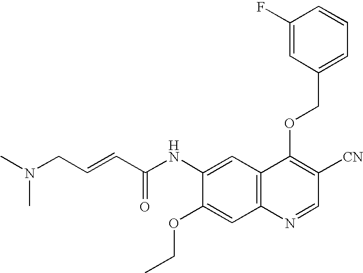 |

Small molecule EGFR inhibitors which can be used in the methods and compositions of the invention include anilinoquinazolines, such as gefitinib, erlotinib, lapatinib, canertinib, vandetanib, and CL-387785 and the other anilinoquinazolines disclosed in PCT Publication No. WO/2005/018677 and U.S. Pat. Nos. 5,747,498 and 5,457,105; quinoline-3-carbonitriles, such as pelitinib, HKI-272, and HKI-357, and the quinoline-3-carbonitriles disclosed in U.S. Pat. Nos. 6,288,082 and 6,002,008; pyrrolopyrimidines, such as PKI166, and the pyrrolopyrimidines disclosed in U.S. Pat. No. 6,713,474 and U.S. Patent Publication Nos. 20060211678, 20060035912, 20050239806, 20050187389, 20050165029, 20050153989, 20050037999, 20030187001, and 20010027197; pyridopyrimidines, such as those disclosed in U.S. Pat. Nos. 5,654,307 and 6,713,484; pyrazolopyrimidines, such as those disclosed in U.S. Pat. Nos. 6,921,763 and 6,660,744 and U.S. Patent Publication Nos. 20060167020, 20060094706, 20050267133, 20050119282, 20040006083, and 20020156081; isoxazoles, such as leflunomide; imidazoloquinazolines, pyrroloquinazolines, and pyrazoloquinazolines. Preferably, the small molecule EGFR inhibitor contains a heterobicyclic or heterotricyclic ring system. Each of the patent publications listed above is incorporated herein by reference.

By “A77 7628” is meant the active metabolite of leflunomide having the structure below.

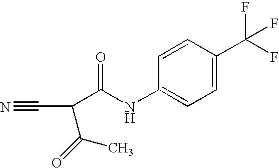

Other features and advantages of the invention will be apparent from the following Detailed Description, the drawings, and the claims.

### BRIEF DESCRIPTION OF THE DRAWINGS

FIG. 1 is a schematic view of a microdermabrasion and drug delivery device.

FIG. 2 is a schematic of an alternative microdermabrasion device in which the device also includes a recycling unit.

### DETAILED DESCRIPTION

The invention features compositions, methods, kits, and devices for administering pharmaceutical compounds to a patient. In general, the invention features the propulsion of particles containing a pharmaceutical compound (e.g., in a controlled release formulation) into a tissue of a patient (e.g., skin). The particles may, for example, be propelled into an intact tissue, or they may be propelled onto a tissue after one or more layers of tissue have been removed. Furthermore, the invention features a device that first removes the stratum corneum, or that continually circulates drug particles (i.e. by propelling them against the skin, then vacuuming out the non-embedded particles, recycling them, and propelling them again) which has the advantage over prior art devices of more accurately controlling the depth of embedding and reducing the amount of drug that is wasted. Further details of the methods, kits, and compositions of the invention are provided below.

### Methods of Drug Delivery

The invention features methods of delivering a therapeutic compound to a tissue by continually propelling particles against the tissue at a velocity sufficient to breach the interface and penetrate into the tissue. The method may involve the steps of (a) removing the most superficial layers of the skin, for example, by abrading the skin with microdermabrasion particles, tape stripping, a chemical peel, or light-based methods, and (b) propelling drug particles at a velocity sufficient to embed a significant percentage (e.g. more than 1%, 5%, 10%, 20%, 30%, 40%, 50%, or more) of the particles into the skin. These steps could also be combined to occur simultaneously in the same procedure.

In one embodiment, a microdermabrasion device is used for, in a first step, removing the stratum corneum of the skin and, in a second step, propelling drug particles at a velocity sufficient to embed a significant percentage (e.g. more than 1%, 2%, 5%, 10%, 20%, 25%, 30%, 40%, 50%, or more) of the particles into the skin. Both steps can be performed using the same device, or optionally with different devices.

While typical sizes needed to attain significant disruption of the skin (at typical propulsion velocities used in microdermabrasion devices) are on the order of 100 micron, ideal sizes for drug delivery particles may be one or more orders of magnitude smaller. Therefore a method of contacting a tissue with particles of two different sizes, such that certain particles disrupt the tissue, while other particles efficiently deliver a pharmaceutical compound to the tissue is desirable. Furthermore, typical dosing needed for skin abrasion is widely different than dosing need for drug delivery. While generally a microdermabrasion procedure uses on the order of 200 g of particles per treatment, typical drug dosages are on the order of mg. Because of the 1,000 to a 100.000-fold difference between the doses needed for abrasion and for drug delivery, the invention features the combination of different doses of drug and abrasive particles. In the first step, any of the types of particle (also in some cases “crystals”) known in the art (e.g. alumina and other metal oxides, glass, salts such as sodium chloride or sodium bicarbonate, ice, or any type of biocompatible particle such as the ones described by Weber et al in U.S. Pat. No. 6,764,493, U.S. Pat. No. 6,726,693, and U.S. Pat. No. 6,306,119) can be propelled against the skin until most or all of the stratum corneum has been removed. Optionally, the debris generated can be vacuumed out to clean the surface of the skin. If metal oxide particles such as alumina are used to perform the abrasion step, the abrasion ideally does not proceed beyond the stratum corneum in order to minimize granuloma formation. If salts, ice, or other biocompatible materials are used, it may be desirable to proceed beyond the stratum corneum and remove portions or all of the epidermis, and portions or all of the dermis. Particles embedded during this first step can be removed by applying water (if, for example, they are particles of salt, ice, or water soluble compounds), or mildly warming the skin (if, for example, the particles have melting points near room temperature). Typically, during the abrasion step, a negligible amount of particles gets embedded into the skin, since few particles have enough momentum density to penetrate the stratum corneum. It has been determined that a momentum density higher than 2.5 kg/(sec*m) is required in order to breach and cross the stratum corneum (Kendall et al, J of Biomechanics, 37, 2004); a typical, 100-micron alumina particle, with density of 3.7 g/cm³, propelled at 30 msec has a momentum density of 1.9 kg/(sec*m), which is insufficient to penetrate the full stratum corneum.

In the second step of the above-described method, drug-containing particles are propelled against the skin using a microdermabrasion device (e.g., the same microdermabrasion device used in the first step), and embed at certain desired depths.

Since the stratum corneum has been removed in the previous step, the remaining skin no longer possesses the mechanical cohesion and integrity of normal skin. Well-controlled penetration of the particles to desired depths can then be ensured by altering one or more of the parameters selected from particle size, particle shape, particle density, and particle velocity. In addition, particle penetration is also a function of the specific characteristics of the skin, which in turn depend on the age of the subject, and on the area of the body being treated. These parameters can be modified by the doctor or practitioner to ensure consistent and desirable penetration for the particular subject and/or tissue being treated. For example, particle density can be increased by compacting the pharmaceutical composition using high pressure and optionally vacuum, as described in WO1997048485. The resulting compacted materials can be attritioned into small particles using conventional methods. Particle velocity can be varied by adjusting the level of vacuum—if a suction pump is used to propel the particles- or the positive pressure level—if a source of compressed air is used to propel the particles. The specific geometry of the device nozzle has an effect on particle velocity, as well. Entrainment of the drug particles by the gas flow occurs in the same manner as entrainment of abrasive crystals. For example, in a compressor-assisted system, air from a compressor is flown through the particle cartridge or a mixing bottle, the air entrains drug particles and the exiting stream is directed to a handpiece.

The methods and devices of the invention extend the range of feasible particle sizes that can be embedded in the skin. Since the resistance to particle penetration is greatly reduced after removal of the stratum corneum, smaller particles can be inserted at a given velocity. For example, 10-micron particles of drug with a density of 1 g/cm³ (a typical value for drug formulations), and with a velocity of 1000 m/sec, would typically not embed because their momentum density is ˜1.7 kg/(sec*m) (below the threshold of 2.5 kg/(sec*m) to cross the stratum corneum). However, after removing the stratum corneum embedding can be achieved.

The penetration depth can be adjusted to anywhere between 0.01 mm and 7 mm. The penetration depth of the particles can be predicted by a penetration model that accounts for the inertial force of the particle and the static force required to yield the skin (Dehn, Int J of Impact Eng, 5, 239-248, 1987). This is given by the relationship:

$$d = \frac{4 {ρ}_{p} {r}_{p}}{3 {ρ}_{t}} \{ ln ( \frac{1}{2} {ρ}_{t} {v}_{i}^{2} + 3 {σ}_{t} ) - ln ( 3 {σ}_{t} ) \}$$

Where d is the penetration depth, ρₚ is the particle density, ρₜ is the tissue density (skin), rₚ is the particle radius, vᵢ is the particle velocity at impact, and, σₜ is the yield stress of the tissue, which in this case corresponds to the skin without stratum corneum. The expression above can be used to guide the design of the drug carrier as well as the selection of operation conditions of the propelling device.

Drug particles may be non-spherical to facilitate embedding and reduce loss by vacuuming. Alternatively, the abrasive particles may be spherical while the drug particles are non-spherical, which facilitates preferential embedding of the drug particles and preferential removal of the abrasive particles.

Any mechanical, chemical, electromagnetic, ultrasound, or light-based method is used to remove the stratum corneum, following which a device selected from the group of a microdermabrasion device and a velocity-based or needle-free transdermal delivery device is used to embed particles into the skin. Also, a mixture of biocompatible abrasive particles and drug particles can be propelled against the skin simultaneously so that the treatment consists of a single step.

Different drug-containing particles can be delivered to different depths in the skin at which their action is desired. The drugs can be applied simultaneously by one single gas jet at a given velocity; in which case their ratio of sizes, their ratio of densities, or their ratio of sphericity determine the difference in penetration depths. The drugs can also be applied in sequential steps, in which case the particles can have different or equal sizes, densities, or shapes, and they can be applied at different velocities. The different drugs may be applied with the purpose of treating different conditions simultaneously, or with the purpose of treating one single condition through a combination therapy of several drugs. A combination therapy with different particle-containing drugs may be helpful in cases where the action of the different drugs takes place at different locations in the skin. By way of example but not by way of limitation, a combination therapy for hair growth could consist of application of minoxidil-containing particles and particles containing inhibitors of steroid metabolism. Minoxidil is thought to work by increasing vascular circulation to the hair follicle, while inhibitors of steroid metabolism affect the hair cycle by stopping the conversion of testosterone to dihydrotestosterone. While a topically applied formulation of minoxidil and an inhibitor of steroid metabolism would distribute everywhere in the skin, application through different particles could be tailored so that the drugs embed preferentially at different depths where they are most effective or where they have the least side effects.

A combination therapy for the treatment of psoriasis could consist of using particles containing corticosteroids (which have an anti-inflammatory action) and particles containing Vitamin D analogues (which reduce lesions by acting on keratinocytes). A combination therapy for acne could consist of using particles containing retinoids (which normalize desquamation of the follicular epithelium) and particles containing antibiotics (which inhibit the growth of P. acnes).

### Microdermabrasion Beads

The compounds of the invention (e.g., EGFR inhibitors) can be formulated into microdermabrasion particles. These particles, when used in a microdermabrasion device, can serve one or more of the following purposes: (1) abrade the skin to a precisely defined depth that optimizes a subsequent treatment for a skin-related condition such as hair follicle regeneration (e.g., EGFR inhibitors), (2) deliver a controlled release formulation of a therapeutic compound, and (3) provide elimination of the therapeutic compound carrier by a natural, or an internally or externally triggered degradation process after the therapeutic compound has been released.

The microdermabrasion particles of the invention may be, for example, 0.05 μm to 200 μm in diameter (e.g., from 15 μm to 150 μm, 0.1 μm to 10 μm, or 1 μm to 2 μm). Particles larger than 150 μm can be used in combination with microdermabrasion devices modified to accommodate larger particles. The ideal average particle diameter and acceptable standard deviation would depend on the condition being treated, the specific therapeutic compound being released, and the desired timing of the release.

In one aspect of the invention, the particle size distribution (psd) of the population of particles will be very narrow. In one aspect, the average particle size is near 100 μm and 90% in weight of the particle composition can pass through a 200 μm mesh screen (preferably, 95%, more preferably, 99%), In one aspect, the particle size distribution is monomodal.

In another aspect of the invention, the microdermabrasion particles provide controlled release (e.g., delayed, sustained, or modified release) of a compound (e.g., an EGFR inhibitor). In particles formulated for delayed release, therapeutic compound may not be substantially released prior to the induction of reepithelialization or prior to a certain phase of reepithelialization, as described below. In one embodiment, an exogenous stimulus is administered to trigger release or activation of the compound, for example, over a period of several seconds, several minutes, several hours, several days (e.g., 2, 3, 4, 5, 6, 7, 8, 9, or 10 days), or several weeks (e.g., 2 weeks) or months. Some examples of exogenous triggers that can be used to stimulate therapeutic compound release include, without limitation, application of light, heat, electricity, magnetism, ultrasound, or chemicals. Alternatively, the therapeutic compound may be designed in a way such that the release is triggered by an endogenous event related to any of the parameters characteristic of the reepithelialization, as described below. Some examples of endogenous triggers are (1) increased expression of a marker that can bind to or enzymatically cleave the particle carrier that contains the therapeutic compound, thereby causing a change in the structure of the carrier particle which enables therapeutic compound release, and (2) increased levels of water in the skin due to completion of the reepithelialization process which causes hydrolytic cleavage of a crosslinked gel structure, or swelling of a hydrogel, thereby allowing therapeutic compound release.

Particles with a specific release window can be designed by manipulating parameters relating to the physical and chemical properties of the carrier, and to a lesser extent by manipulating the concentration of additives such as emulsifiers. If the carrier is a polymer, the molecular weight, hydrophilicity, and relative ratios of the monomers of the polymer (in the case of a copolymer) can be tailored so that a specific release window is obtained. Several polymers with different degradation kinetics may coexist in one formulation; in this case, the total rate of release is the average of the rates of release from each polymer, which can therefore be tuned by adjusting the ratio of polymers in the formulation, as described, for example, in U.S. Pat. No. 4,897,268.

Well-controlled synthesis methods known in the art (e.g., those described below) may be used to generate particles with the controlled release and disruption properties described above. The synthesis methods include, but are not limited to, coacervation, emulsion phase separation, spray-drying encapsulation, and solvent evaporation in organic or water phase. These methods are well known in the art, and have been described in, for example, U.S. Pat. No. 6,506,410. In one embodiment, synthesis may involve solvent evaporation in water phase. Solvent evaporation can follow water/oil/water emulsification, which is used to encapsulate water-soluble therapeutic compounds (for example, in a biodegradable carrier), or oil/water encapsulation, for lipid-soluble therapeutic compounds. A feature of this method is that the oil/water technique yields particles that are more porous, allowing a high burst of therapeutic compound to be delivered initially (Ibid). Therapeutic compound-loaded particles may also be produced by dispersing porous carrier particles into a solution containing the therapeutic compound, whereby the therapeutic compound in solution penetrates the pores of the carrier and remains trapped inside. In a subsequent step, an additive can be added to facilitate stabilization of the therapeutic compound in the carrier. The fluid in the remaining solution may then be separated by decantation, drying, lyophilization, vacuum-drying, or other methods known to people skilled in the art.

Some examples of polymers that may be synthesized by these methods include cellulose derivatives (e.g. cellulose acetate, cellulose butyrate, ethyl cellulose), poly(urethanes), poly(siloxanes), poly(carbonates), poly(butadienes), poly(esters), poly(hydroxybutyric acid), poly(methyl methacrylate), poly(vinyl acetate), poly(vinyl alcohol), poly(ethylenes), waxes, proteins, and lipids (Ibid). In certain embodiments, the carrier may be chemically inert, non-degradable, and processable by the synthesis methods described above. Some materials with such properties that are well-suited for controlled release include poly(2-hydroxy ethyl methacrylate), poly(N-vinyl pyrrolidone), poly(methyl methacrylate), poly(vinyl alcohol), poly(acrylic acid), poly(acrylamide), poly(ethylene-co-vinyl acetate), poly(ethylene glycol), and poly(methacrylic acid). In certain other embodiments, the carrier may be designed so that it degrades within the body, while still being processable by the synthesis methods described above. Some materials that are biodegradable and well-suited for controlled release include poly(lactides), poly(glycolides), poly(lactide-go-glycolides), poly(anhydrides), and poly(orthoesters). In certain other embodiments, the carrier may be designed so that some important property, such as phase state or swelling, changes at or close to body temperature. Materials that are solid slightly below body temperature but that melt at or slightly above body temperature include low melting fats, and mixtures of low melting and high melting fats. Materials that swell around body temperature include temperature-sensitive hydrogels. In certain other embodiments, the carrier may be designed so that it adheres strongly to the skin. Materials suited for this purpose tend to have high concentration of polar groups (i.e. carboxylic acid), high molecular weight, polymer chain flexibility, and surface charge. (e.g., poly(anhydride)). In certain other embodiments, the carrier material may be designed such that the release of the therapeutic compound may be triggered by an exogenous or endogenous event. For example, exposure to UV light can cause photorelease of a therapeutic compound in poly(amides); ultrasound can accelerate therapeutic compound release from poly(anhydrides); hydrogels can be designed so that changes in temperature, pH, ionic strength, or binding of certain molecules trigger the therapeutic compound release. In certain other embodiments, the carrier may be designed so that the therapeutic compound is released at a constant rate. Carriers with such properties include double-walled polymer systems, such as a mixture of poly(1,3-bis(p-carboxyphenoxypropane)-co-sebacic anhydride and poly(lactic acid).

### Antioxidants

If desired, the small molecule therapeutic compound (e.g, EGFR inhibitor) formulations of the invention can contain one or more antioxidants. Useful antioxidants include, without limitation, thiols (e.g., aurothioglucose, dihydrolipoic acid, propylthiouracil, thioredoxin, glutathione, cysteine, cystine, cystamine, thiodipropionic acid), sulphoximines (e.g., buthionine-sulphoximines, homo-cysteine-sulphoximine, buthionine-sulphones, and penta-, hexa- and heptathionine-sulphoximine), metal chelators (e.g, α-hydroxy-fatty acids, palmitic acid, phytic acid, lactoferrin, citric acid, lactic acid, and malic acid, humic acid, bile acid, bile extracts, bilirubin, biliverdin, EDTA, EGTA, and DTPA), vitamins (e.g., vitamin E, vitamin C, ascorbyl palmitate, Mg ascorbyl phosphate, and ascorbyl acetate), phenols (e.g., butylhydroxytoluene, butylhydroxyanisole, ubiquinol, nordihydroguaiaretic acid, trihydroxybutyrophenone), benzoates (e.g., coniferyl benzoate), uric acid, mannose, propyl gallate, selenium (e.g., selenium-methionine), stilbenes (e.g., stilbene oxide and trans-stilbene oxide), and combinations thereof.

Antioxidants that may be incorporated into the formulations of the invention include natural antioxidants prepared from plant extracts, such as extracts from aloe vera; avocado; chamomile; echinacea; ginko biloba; ginseng; green tea; heather; jojoba; lavender; lemon grass; licorice; mallow; oats; peppermint; St. John's wort; willow; wintergreen; wheat wild yam extract; marine extracts; and mixtures thereof.

The total amount of antioxidant included in the formulations can be from 0.001% to 3% by weight, preferably 0.01% to 1% by weight, in particular 0.05% to 0.5% by weight, based on the total weight of the formulation.

### Other Biologically Active Ingredients

Other biologically active agents that can be used in the methods, kits, and compositions of the invention include, without limitation, antihistamines, anti-inflammatory agents, anti-cancer agents, retinoids, anti-androgen agents, immunosuppressants, channel openers, antimicrobials, herbs (e.g., saw palmetto), extracts (e.g., Souhakuhi extract), vitamins (e.g., biotin), co-factors, psoralen, anthralin, and antibiotics.

### Antihistamines

In certain embodiments, an antihistamine can be used in the compositions, methods, and kits of the invention. Useful antihistamines include, without limitation, Ethanolamines (e.g., bromodiphenhydramine, carbinoxamine, clemastine, dimenhydrinate, diphenhydramine, diphenylpyraline, and doxylamine); Ethylenediamines (e.g., pheniramine, pyrilamine, tripelennamine, and triprolidine); Phenothiazines (e.g., diethazine, ethopropazine, methdilazine, promethazine, thiethylperazine, and trimeprazine); Alkylamines (e.g., acrivastine, brompheniramine, chlorpheniramine, desbrompheniramine, dexchlorpheniramine, pyrrobutamine, and triprolidine); piperazines (e.g., buclizine, cetirizine, chlorcyclizine, cyclizine, meclizine, hydroxyzine); Piperidines (e.g., astemizole, azatadine, cyproheptadine, desloratadine, fexofenadine, loratadine, ketotifen, olopatadine, phenindamine, and terfenadine); and Atypical antihistamines (e.g., azelastine, levocabastine, methapyrilene, and phenyltoxamine). Both non-sedating and sedating antihistamines may be employed. Non-sedating antihistamines include loratadine and desloratadine. Sedating antihistamines include azatadine, bromodiphenhydramine; chlorpheniramine; clemizole; cyproheptadine; dimenhydrinate; diphenhydramine; doxylamine; meclizine; promethazine; pyrilamine; thiethylperazine; and tripelennamine.

Other antihistamines suitable for use in the compositions, methods, and kits of the invention are acrivastine; ahistan; antazoline; astemizole; azelastine; bamipine; bepotastine; bietanautine; brompheniramine; carbinoxamine; cetirizine; cetoxime; chlorocyclizine; chloropyramine; chlorothen; chlorphenoxamine; cinnarizine; clemastine; clobenzepam; clobenztropine; clocinizine; cyclizine; deptropine; dexchlorpheniramine; dexchlorpheniramine maleate; diphenylpyraline; doxepin; ebastine; embramine; emedastine; epinastine; etymemazine hydrochloride; fexofenadine; histapyrrodine; hydroxyzine; isopromethazine; isothipendyl; levocabastine; mebhydroline; mequitazine; methafurylene; methapyrilene; metron; mizolastine; olapatadine; orphenadrine; phenindamine; pheniramine; phenyltoloxamine; p-methyldiphenhydramine; pyrrobutamine; setastine; talastine; terfenadine; thenyldiamine; thiazinamium; thonzylamine hydrochloride; tolpropamine; triprolidine; and tritoqualine.

Antihistamine analogs can be used in the compositions, methods, and kits of the invention. Antihistamine analogs include 10-piperazinylpropylphenothiazine; 4-(3-(2-chlorophenothiazin-10-yl)propyl)-1-piperazineethanol dihydrochloride; 1-(10-(3-(4-methyl-1-piperazinyl)propyl)-10H-phenothiazin-2-yl)-(9CI) 1-propanone; 3-methoxycyproheptadine; 4-(3-(2-Chloro-10H-phenothiazin-10-yl)propyl)piperazine-1-ethanol hydrochloride; 10,11-dihydro-5-(3-(4-ethoxycarbonyl-4-phenylpiperidino)propylidene)-5H-dibenzo(a,d)cycloheptene; aceprometazine; acetophenazine; alimemazin (e.g., alimemazin hydrochloride); aminopromazine; benzimidazole; butaperazine; carfenazine; chlorfenethazine; chlormidazole; cinprazole; desmethylastemizole; desmethylcyproheptadine; diethazine (e.g., diethazine hydrochloride); ethopropazine (e.g., ethopropazine hydrochloride); 2-(p-bromophenyl-(p′-tolyl)methoxy)-N,N-dimethyl-ethylamine hydrochloride; N,N-dimethyl-2-(diphenylmethoxy)-ethylamine methylbromide; EX-10-542A; fenethazine; fuprazole; methyl 10-(3-(4-methyl-1-piperazinyl)propyl)phenothiazin-2-yl ketone; lerisetron; medrylamine; mesoridazine; methylpromazine; N-desmethylpromethazine; nilprazole; northioridazine; perphenazine (e.g., perphenazine enanthate); 10-(3-dimethylaminopropyl)-2-methylthio-phenothiazine; 4-(dibenzo(b,e)thiepin-6(11H)-ylidene)-1-methyl-piperidine hydrochloride; prochlorperazine; promazine; propiomazine (e.g., propiomazine hydrochloride); rotoxamine; rupatadine; Sch 37370; Sch 434; tecastemizole; thiazinamium; thiopropazate; thioridazine (e.g., thioridazine hydrochloride); and 3-(10,11-dihydro-5H-dibenzo(a,d)cyclohepten-5-ylidene)-tropane.

Other compounds that are suitable for use in the compositions, methods, and kits of the invention are AD-0261; AHR-5333; alinastine; arpromidine; ATI-19000; bermastine; bilastin; Bron-12; carebastine; chlorphenamine; clofurenadine; corsym; DF-1105501; DF-11062; DF-1111301; EL-301; elbanizine; F-7946T; F-9505; HE-90481; HE-90512; hivenyl; HSR-609; icotidine; KAA-276; KY-234; lamiakast; LAS-36509; LAS-36674; levocetirizine; levoprotiline; metoclopramide; NIP-531; noberastine; oxatomide; PR-881-884A; quisultazine; rocastine; selenotifen; SK&F-94461; SODAS-HC; tagorizine; TAK-427; temelastine; UCB-34742; UCB-35440; VUF-K-8707; Wy-49051; and ZCR-2060.

Still other compounds that can be used in the compositions, methods, and kits of the invention are described in U.S. Pat. Nos. 3,956,296; 4,254,129; 4,254,130; 4,282,233; 4,283,408; 4,362,736; 4,394,508; 4,285,957; 4,285,958; 4,440,933; 4,510,309; 4,550,116; 4,692,456; 4,742,175; 4,833,138; 4,908,372; 5,204,249; 5,375,693; 5,578,610; 5,581,011; 5,589,487; 5,663,412; 5,994,549; 6,201,124; and 6,458,958.

### Antimicrobial Agents

In certain embodiments, an antimicrobial agent can be used in the compositions, methods, and kits of the invention. Useful antimicrobial agents include, without limitation, benzyl benzoate, benzalkonium chloride, benzoic acid, benzyl alcohol, butylparaben, ethylparaben, methylparaben, propylparaben, camphorated metacresol, camphorated phenol, hexylresorcinol, methylbenzethonium chloride, cetrimide, chlorhexidine, chlorobutanol, chlorocresol, cresol, glycerin, imidurea, phenol, phenoxyethanol, phenylethylalcohol, phenylmercuric acetate, phenylmercuric borate, phenylmercuric nitrate, potassium sorbate, sodium benzoate, sodium proprionate, sorbic acid, and thiomersal.

The antimicrobial can be from about 0.05% to 0.5% by weight of the total composition, except for camphorated phenol and camphorated metacresol. For camphorated phenol, the preferred weight percentages are about 8% to 12% camphor and about 3% to 7% phenol. For camphorated metacresol, the preferred weight percentages are about 3% to 12% camphor and about 1% to 4% metacresol.

### Anti-Inflammatory Agents

In certain embodiments, an antiinflammtory agent can be used in the compositions, methods, and kits of the invention. Useful antiinflammtory agents include, without limitation, Non-Steroidal Anti-Inflammatory Drugs (NSAIDs) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), and corticosteroids (e.g., alclometasone dipropionate, amcinonide, betamethasone dipropionate, betamethasone valerate, clobetasol propionate, desonide, desoximetasone, dexamethasone, diflorasone diacetate, flucinolone acetonide, flumethasone, fluocinonide, flurandrenolide, halcinonide, halobetasol propionate, hydrocortisone butyrate, hydrocortisone valerate, methylprednisolone, mometasone furoate, prednisolone, or triamcinolone acetonide).

### Immunosuppressants

In certain embodiments, a nonsteroidal immunosuppressant can be used in the compositions, methods, and kits of the invention. Suitable immunosuppressants include cyclosporine, tacrolimus, rapamycin, everolimus, and pimecrolimus.

### Cyclosporines

The cyclosporines are fungal metabolites that comprise a class of cyclic oligopeptides that act as immunosuppressants. Cyclosporine A is a hydrophobic cyclic polypeptide consisting of eleven amino acids. It binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T cell-dependent immune response by inhibiting antigen-triggered signal transduction. This inhibition decreases the expression of proinflammatory cytokines, such as IL-2.

Many different cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Application Publication No. 2002/0132763 A1). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala(3-acetylamino)-8-Cs, Thr-2-Cs, and D-MeSer-3-Cs, D-Ser(O—CH₂CH₂—OH)-8-Cs, and D-Ser-8-Cs, which are described in Cruz et al., Antimicrob. Agents Chemother. 44:143 (2000).

### Tacrolimus

Tacrolimus and tacrolimus analogs are described by Tanaka et al. (J. Am. Chem. Soc., 109:5031 (1987)) and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

### Pimecrolimus

Pimecrolimus is the 33-epi-chloro derivative of the macrolactam ascomyin. Pimecrolimus structural and functional analogs are described in U.S. Pat. No. 6,384,073.

### Rapamycin

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

### Retinoids

In certain embodiments, a retinoid can be used in the compositions, methods, and kits of the invention. Useful retinoids include, without limitation, 13-cis-retinoic acid, 9-cis retinoic acid, all-trans-retinoic acid, etretinate, acitretin, retinol, retinal, tretinoin, alitretinoin, isotretinoin, tazarotene, bexarotene, and adapelene.

### Channel Openers

In certain embodiments, a channel opener can be used in the compositions, methods, and kits of the invention. Useful channel openers include, without limitation, minoxidil, diazoxide, and phenyloin.

### Anti-Androgens

In certain embodiments, an anti-androgen can be used in the compositions, methods, and kits of the invention. Useful anti-androgens include, without limitation, finasteride, flutamide, diazoxide, 11alpha-hydroxyprogesterone, ketoconazole, RU58841, dutasteride, fluridil, QLT-7704, and anti-androgen oligonucleotides.

### Antibiotics

In certain embodiments, an antibiotic can be used in the compositions, methods, and kits of the invention. Useful antibiotics include, without limitation, penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmatozole, cefotaxime, ceftizoxime, ceftriaxone, cefoperazone, ceftazidime, cefixime, cefpodoxime, ceftibuten, cefdinir, cefpirome, cefepime, BAL5788, BAL9141, imipenem, ertapenem, meropenem, astreonam, clavulanate, sulbactam, tazobactam, streptomycin, neomycin, kanamycin, paromycin, gentamicin, tobramycin, amikacin, netilmicin, spectinomycin, sisomicin, dibekalin, isepamicin, tetracycline, chlortetracycline, demeclocycline, minocycline, oxytetracycline, methacycline, doxycycline, erythromycin, azithromycin, clarithromycin, telithromycin, ABT-773, lincomycin, clindamycin, vancomycin, oritavancin, dalbavancin, teicoplanin, quinupristin and dalfopristin, sulphanilamide, para-aminobenzoic acid, sulfadiazine, sulfisoxazole, sulfamethoxazole, sulfathalidine, linezolid, nalidixic acid, oxolinic acid, norfloxacin, perfloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, sitafloxacin, metronidazole, daptomycin, garenoxacin, ramoplanin, faropenem, polymyxin, tigecycline, AZD2563, and trimethoprim.

### Growth Factors

In another embodiment, growth factors, growth factor antagonists, and growth factor agonists, can also be used in the compounds of the invention.

### Reepithelialization

In one aspect of this invention, the compositions of the invention are administered and released into a subject's skin (without limitation examples of the skin location are the head, for example, the scalp, the face, the eyebrow, or a scarred region) while the skin is in a state of reepithelialization. Reepithelialization is the process that occurs during formation of a new epidermis and can be characterized for the purposes of this invention by the lack of fully formed hair follicles (e.g., if within the tissue some cells are in the pre-placode stage of hair follicle formation), an embryonic-like state, in which the follicle regenerates, or by lack of a stratum corneum.

### State of Reepithelialization

Reepithelialization can be detected through inspection of the new epidermis where covering of the wound area by keratinocytes indicates reepithelialization. The presence of keratinocytes can be seen with the naked eye as a white, glossy, shiny surface that gradually covers the open wound. Using a confocal microscope, keratinocytes can be visualized as a sheet of “cobblestone”-looking cells. Reepithelialization can also be detected through the measurement of transepidermal water loss (TEWL). TEWL decreases when the epithelial barrier is restored. Confocal scanning laser microscopy and/or optical coherence tomography can also be used to detect the state of reepithelialization, where the presence of keratinocytes indicates reepithelialization.

The presence of a stratum corneum can be determined though visual inspection, direct observation of papillary blood vessels using a capillary microscope, or through a colorimetric redox reaction of a compound that reacts in the presence of live cells. For example, 0.01% nitrazine yellow applied to the skin will remain yellow if a stratum corneum is present, and will turn greenish brown if not. In another example 0.01% bromcresol purple applied to the skin will stay yellow if the stratum corneum is present and will turn purple if the stratum corneum is not present.

The area of reepithelialization can be, for example, between 0-2 centimeters (cm) in width (e.g., 1 cm, 1.5 cm, and 2.0 cm) or the area may be greater. Optionally, the area of reepithelialization can be interfollicular (e.g., the area of disruption leading to reepithelialization can be limited to the area immediately surrounding the previously existing or new follicle).

In some aspects of the invention, it is desirable to release the compounds of the invention at a particular phase of reepithelialization. Stages at which compounds of the invention may preferably be administered and/or activated include periods:
prior to disruption,
simultaneous with disruption,
after completion of the reepithelialization process (e.g., 3-12 days, or 9-11 days after having disrupted the skin),
after or during the establishment of a stem cell population that will develop into a regenerated hair follicle (Ito et al, Nature 447, 316-320, May 2007),
prior to the expression of hair follicle differentiation markers KRT17 and Lef1 for several days after wound closure (Ito et al, Nature 447, 316-320, May 2007),
after or during expression of one or more proteins including KRT17, Lef-1, alkaline phosphatase, Wnt10b, and Shh (Ito et al, Nature 447, 316-320, May 2007),
characterized by the absence of K10 expression (which is expressed in normal epidermis) and/or induction of expression of K16 and K17 (which are not expressed in normal epidermis) (Patel et al, Journal of Investigative Dermatology, 126, 2006),
charactized by the elevation of one or more transcription factors including AP-1 and NF-κB, primary cytokines IL-10 and TNF-α, and matrix metalloproteases (Karimipour et al, Journal of the American Academy of Dermatology, 52, Issue 2, 2005),
characterized by histologic changes (Freedman et al, Dermatologic Surgery, 27 Issue 12, December 2001), including, for example:
   thickening of the epidermis and dermis,
   flattening of rete pegs,
   vascular ectasia,
   perivascular inflammation,
   hyalinization of the papillary dermis with newly deposited collagen and elastic fibers,
   change in orientation, density, or packing of collagen and other structures,
characterized by detachment of the scab. Depending on the depth of the abrasion process, it may be desirable for the compounds of the invention to be administered or activated prior to or after the detachment of a scab. Alternatively, hair follicles may start to form before the scab falls off, in the case of, for example, dermabrasion.

Alternatively the compounds of the invention can be administered prior to epidermal disruption. In such embodiments, the compound may be formulated for controlled release such that the therapeutically active compound is released during reepithelialization or during a particular phase of reepithelialization (e.g., as described above). The compound may also be formulated such that it becomes activated by an endogenous or exogenous stimulus (e.g., as described below).

### Induction of Reepithelialization

The state of reepithelialization can be induced. Methods of inducing this state include the disruption of the subject's skin at the location where the compounds of the invention are going to be administered. Disruption can be achieved through abrasion (e.g., the rubbing or wearing away of skin), or through any method that results in disturbing the intactness of the epidermis or epidermal layer including burning (e.g., by inducing a sunburn) or perforating the epidermis or epidermal layer. The disruption can either result in partial or complete removal of the epidermal layer at the intended location.

The disruption of the epithelial layer can be accomplished, for example, through mechanical, chemical, electromagnetic, or electrical means. Mechanical means can be achieved through the use of, for example, sandpaper, a felt wheel, ultrasound, supersonically accelerated mixture of saline and oxygen, tape-stripping, microdermabrasion, or application of chemical compounds (e.g. peels)

Microdermabrasion provides a way of disrupting (e.g., abrading) skin to an optimal depth simultaneous with, or followed by, application of particles (or lotion, gel, cream, or foam) that can release a therapeutic compound in a sustained and/or controlled release manner over a window that is relevant to hair follicle regeneration.

In one particular example, the particles are suspended in a fluid (e.g. liquid or gas) and projected through a tip to the tissue being treated, for example, as described previously (e.g., U.S. Pat. No. 5,037,432). The particles projected on the skin first disrupt the superficial tissue to a certain depth (e.g., as described below). The particles either stay on the surface of the disrupted skin (and may subsequently be removed) or become inserted into the skin. Immediately after wounding or after a certain delay as described above, the therapeutic compound contained in the particles is released either immediately or in a controlled manner over a period of hours to weeks. If a carrier was used to deliver the therapeutic compound, the carrier becomes biodegraded and cleared from the skin naturally or by a degradation process triggered exogenously or endogenously. The skin debris and the abrading particles left on the surface of the skin after or during the initial wounding step may be removed with a vacuum.

The depth of the abrasion performed on the skin may be optimized to achieve maximum hair follicle regeneration or another therapeutic benefit. The particles described in this invention may be used to abrade the skin to narrowly defined depths, from a minimum of 5 μm, which only removes partially the stratum corneum, to a maximum of about 5 mm, which completely removes the dermis. A given depth may be achieved by (i) varying the particle velocity and flow rate (e.g., by adjusting the level of the suction pressure that draws the particles out of the cartridge in the microdermabrasion device), and/or (ii) adjusting the number of times the device is passed over the skin.

Any of the above described methods may be used to remove a precise amount of epidermal tissue. For example, the methods of abrasion described herein may be used to achieve:
Removal of the stratum corneum through removal of the first 10-30 μm of dead skin cells.
Removal of the stratum corneum and part or all of the epidermis by removing the first 30-100 μm of the skin. This is not deep enough to remove the sebaceous gland, bulge, or hair papilla of existing follicle structures.
Removal of the stratum corneum, the full epidermis, and part of the dermis down to approximately 500 μm. This process removes most of the sebaceous glands, which are at a depth beneath 500 μm.
Removal of the stratum corneum, the full epidermis, and part of the dermis down to approximately 800 μm. This process removes most of the sebaceous glands, and the bulge regions, which are at a depth beneath 800 μm. (Dunkin et al., Plastic Reconstructive Surgery, 119 (6), May 2007)
Removal of the stratum corneum, the full epidermis, and part of the dermis down to approximately 2000-4000 μm. This process removes the sebaceous glands, the bulge regions, and most of the hair papillas, which are at a depth beneath 2000 μm.
Removal of the stratum corneum, the full epidermis, and the full dermis resulting in removal of up to 5 to 7 mm of skin. This process removes all the structures of the follicles, including the sebaceous gland, bulge, and papilla.

In any of the above methods, the disruption can be localized to a region approximately the size of a hair follicle (e.g., the disruption may cover an area of the 0.001 mm², 0.01 mm², 0.05 mm², 0.1 mm², 0.5 mm², 1 mm², 2 mm², 3 mm², 4 mm², or 5 mm²). In such cases, the areas of disruption may be separated from each other. Limiting the area of the disruption may allow deeper disruption without resulting in scar formation. This could be accomplished manually or by, for example, placing a patterned template on the surface of the skin prior to the disruption step, whereby the skin beneath the solid portion of the template is not disrupted and the skin beneath the void portion of the template is exposed to the microdermabrasion and disrupted. For example a mesh or checkerboard template comprised of a thin and flexible but microdermabrasion-resistant material with a series of holes or gaps could be placed on the skin during the microdermabrasion process.

The invention also may feature the recycling of compounds administered as microdermabrasion particles. Not all of the particles that are projected onto the skin will become embedded in it. A significant portion of the particles may remain mixed with the skin debris that may be removed by vacuum. The method to allow recycling of the therapeutic compound or therapeutic compound particles involves the addition of at least one collection or separation operation to the existing microdermabrasion devices. The purpose of this separation operation would be to separate out a fraction of the therapeutic compound or the therapeutic compound and its carrier bead from the remaining materials. Several differential properties between the therapeutic compound (or the therapeutic compound carrier bead) and the other byproducts may be exploited to achieve this separation, including, but not limited to: size, density, solubility, ignition points, vaporization point, melting points, freezing points, ionic properties, magnetic properties, and phase state. The specific separation techniques that would exploit these differential properties include, without limitation, sieving or membrane separation, centrifugation, sedimentation or decantation, burning, vaporization, any type of ionic or affinity separation, magnetic separation, melting, freezing, crystallization, or flocculation. The purpose of a collection step would be to allow the later separation of the therapeutic compound or therapeutic compound particles from the debris, either on or off site.

The invention also features devices for administering the microdermabrasion particles and abrading the skin. Such a device includes a propulsion unit, a handpiece, a tip, and a cartridge or pair of cartridges. The cartridge or cartridges are selected, for example, from a cartridge containing a mixture of abrasive particles and therapeutic compound particles, a cartridge containing therapeutic compound particles formulated into an abrasive solid carrier, and the combination of a cartridge containing abrasive particles and a cartridge containing therapeutic compound particles. Such a device would also optionally include a vacuum source to remove the abraded skin debris, and a recycling unit to separate vacuumed therapeutic compound particles from skin debris and other particles not containing recoverable therapeutic compound, or a collection unit to collect vacuumed therapeutic compound particles and skin particles.

The invention also features a kit for use with a standard microdermabrasion device or with a microdermabrasion device of the invention (e.g., as described above). This kit contains a cartridge of the therapeutic compound containing microdermabrasion particles of the invention, a tip, and, optionally, a recycling unit to separate therapeutic compound from other byproducts of the abrasion step or a collection unit to collect vacuumed therapeutic compound particles and debris for return to the manufacturer and later separation. In one embodiment, the recycling unit can be part of the tip. (e.g. a tip with a sieve incorporated on the section that vacuums the byproducts of the abrasion, so that only certain sizes are allowed back into the device).

Other means of disruption include chemical which can be achieved, for example, using phenol, trichloracetic acid, or ascorbic acid.

Electromagnetic means of disruption of the epidermis can be achieved, for example, by the use of a laser capable of inducing trans-epithelial injury (e.g., a Fraxel laser, a CO₂ laser, or an excimer laser). Disruption can also be achieved through, for example, the use of visible, infrared, ultraviolet, radio, or X-ray irradiation.

Electrical means of disruption of the epidermis can be achieved, for example, through the application of an electrical current or through electroporation.

Any of the previously mentioned means of disruption can be used to induce for example, a burn, excision, or microdermabrasion.

Optionally, the skin, following the epidermal disruption, is not contacted for a period of time with any substance (e.g., ointment, a bandage, or a device) that is normally administered to an abrasion or wound to prevent infection. Here the skin is not contacted with any substance until, for example, the epidermal disruption has healed (e.g., any time between 2 days and 3 weeks). Alternatively, the skin can be contacted with a cast or bandage (e.g., resulting in increased blood flow to the disrupted skin or decreased transdermal water loss or decreased mass transfer of gases into the skin and from the skin (e.g. oxygen, carbon dioxide, water vapor) or decreased heat transfer from the skin (e.g. resulting in an increased temperature of the skin surface).

Prior to disruption, the skin can be depilated or epilated. The depilation or epilation can be accomplished through, for example, waxing, plucking, an abrasive material, a laser, electrolosis, a mechanical device, or thioglycolic acid.

The disruption of the epidermis can be induced between simultaneous with, or 1-12 days (e.g., 4-12, 5-12, 4-11, 6-11, 6-10, 6-9, 7-8, 5-11, 5-10, or 7-10 days) prior to the addition of the compositions of the invention. In another embodiment, the compositions of the invention can be embedded into the skin prior to the disruption of the skin.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1

### Composition for Skin Abrasion, Strong Adhesion to Skin, Controlled Drug Release, Biodegradability, and Use in a Conventional Microdermabrasion Device

One or more of the compounds of the invention (e.g., EGFR inhibitors as described above) are formulated into a polyanhydride polymer synthesized by established methods (Mathiowitz et al, Biomaterials, 24, 2003). This method comprises a first step in which fumaric anhydride oligomer and sebacic anhydride oligomer are blended in a melt polycondensation process, and a second step in which microspheres of this polymer are obtained through a holt melt technique (Mathiowitz et al, J Control Rel, 5, 1987). The spheres obtained are sieved to a certain desired size range (e.g., from 100 to 125 μm). The desired size range may vary depending on (1) the desired release duration (larger particles take longer to degrade and therefore release drug for a longer period), and (2) the desired abrasive power (larger particles are more abrasive).

The poly(anhydride) carrier obtained by this synthesis method has the convenient properties of being rigid, eroding in a biological environment, and adhering strongly to the skin. Rigidity is convenient for the carrier to be abrasive. Erosion in a biological environment allows controlled release of the drug contained in the carrier and clearance of the carrier from the skin after the release is complete. Strong adhesion to the skin makes the carrier less likely to be removed by vacuum than the remaining byproducts of the skin abrasion step (e.g. skin debris). Strong adhesion also ensures that the carrier will remain in contact with the tissue where the drug must be delivered for the desired period of time. The surface roughness and the strength of adhesion of the carrier can be increased by increasing the percentage of fumaric anhydride oligomers in the initial blend.

The carrier particles are packaged in a cartridge that can be slotted into a conventional microdermabrasion device. The cartridge is connected to a suction line that aspirates the particles and propels them at high velocity through a handheld piece. The handheld piece has a tip at its end and includes an outlet through which the propelled particles exit and impact the skin and an inlet through which a vacuum is applied to remove the products of the abrasion step. In another embodiment, the tip may have an outlet for exiting particles and an adjustable inlet to control the strength of the vacuum. In yet another embodiment, the tip may only have an outlet for exiting particles but no inlet for vacuuming. In another embodiment, the tip may have several outlets that allow several types of particles to be propelled against the skin simultaneously. The mixture of products generated by the abrasion step (skin debris and particles) may be removed by applying a vacuum. The vacuumed products may be directed to a separation unit where the particle carrier containing the drug or only the drug contained in the particle carrier are recovered by one of the methods described in herein. In another embodiment, the mixture of products generated by the abrasion step is not vacuumed and remains on the surface of the skin.

In a further embodiment, the pressure is high enough to propel the particles, or a liquid jet containing suspended particles, at velocities that ensure insertion into the epidermis or dermis. Insertion depths on the order of hundreds of μm can be obtained (Mitragorti et al, PNAS, 104(11), 2007). The penetration depth of the particles into the epidermis and dermis can be precisely adjusted so that the highest concentration of particles is at the level of a relevant structure. Relevant insertion depths are, for example, between 10-30 μm (up to or past the stratum corneum), around 100 μm (past the epidermis), between 300-500 μm (past the sebaceous gland), between 500-800 μm (past the bulge), and between 2000-4000 μm (past the papilla). In a preferred embodiment, particles are propelled against the skin after the stratum corneum has been removed (e.g., by conventional microdermabrasion with alumina particles). Inserting the particles into the skin ensures that the majority are not removed by vacuum or mechanical friction. In a preferred embodiment, the average particle diameter is less than 100 μm. In another embodiment, the particles are inserted into the skin by other methods such as ultrasound or injection with microneedles.

A desired depth of abrasion is obtained by adjusting (1) the number of abrasion passes performed with the handholdable piece, (2) the pressure head used to propel the particles at a given velocity and flowrate, and (3) the particle size. In another embodiment, a desired depth of abrasion is obtained by propelling against the skin common abrasives used in microdermabrasion (e.g. alumina particles) simultaneously with particles of drug carrier, prior to the application of particles of drug carrier, or after the application of particles of drug carrier. In another embodiment, a desired depth of abrasion is obtained by propelling against the skin common abrasive particles, such as alumina, which are formulated to contain a drug or a drug and a carrier.

A precise desired duration of drug release can be obtained by using particles with a narrow size distribution. Poly(anhydride) copolymers have been shown to display nearly constant degradation rates and drug release rates at relevant time scales (2 to 15 days) under physiological conditions (Domb et al, Journal of Polymer Science Part A-Polymer Chemistry, 29 (4), 1991).

A constant (or zero order) release of the drug can be obtained by using particles with a double-wall structure. Such a structure consists of an inner core of a first material which contains the drug, surrounded by a shell of a second material which controls the rate of release of the drug. The outer shell does not rapidly degrade, and therefore its thickness remains constant; and as a result, the diffusion rate of the drug is constant as long as there is drug left within the shell. Methods for synthesizing double-walled spheres of poly(anhydride) and poly(lactic acid) have been reported (see, for example, Matthiowitz et al, Nature, 367 (6460), 1994). Such particles can be synthesized by introducing a two-polymer solution of poly(1,3-bis(p-carboxyphenoxypropane)-co-sebacic anhydride and poly(lactic acid) into a continuous phase. A stable emulsion is created, in which phase separation occurs within each drop so that one polymer engulfs the other, thereby forming a double-walled microsphere. Spheres from 20 to 1000 μm with external layers of poly(lactic acid) have been obtained using this method. These can later be sieved so that the range of sizes fall within given acceptable limits. Other synthesis methods, such as solvent evaporation, have been presented elsewhere (Matthiowitz et al, Advanced Materials, 6 (9), 1994). The specific duration of the release may be adjusted by manipulating the sizes of the core and shell.

In another embodiment, the degradation rate of the polyanhydride particles is increased by using ultrasound to a level that does not compromise the integrity of cells. Optional additional features of this embodiment include a permeation enhancer, the combination of the anhydride oligomers with a second polymer (e.g., a poly(styrene), which confers a desired property (e.g., slower drug release), a carrier polymer with favorable bioadhesion properties such as a high concentration of polar groups (e.g. carboxylic acid), high molecular weight, and high surface charge. Examples of polymers with such properties include hydrogels, and hydrophilic polymers containing carboxylic groups such as poly(acrylic acid).

### Example 2

### Composition Sensitive to Exogenous or Endogenous Stimuli for Controlled Drug Release with an Initial Delay, Biodegradability, and Use in a Conventional Microdermabrasion Device

One or more of the pharmaceutical compounds used in the invention (e.g., a small molecule EGFR inhibitor) may also be formulated into a hydrogel using methods known in the art (see, for example, N Peppas et al, J Biomater Sci Polymer Edn, 15, 2, 2004). This hydrogel can swell without dissolving when placed in a biological tissue. The hydrogel carrier has the convenient properties of being degradable in a biological environment, and most notably, the ability to swell in response to changes in the surrounding environment, which in turn may allow the pharmaceutical compound release in a controlled manner. Depending on the specific type of hydrogel, the environmental change that causes the swelling may include a change in pH (acidic or basic hydrogels), temperature (thermoresponsive hydrogel), or ionic strength (ionic hydrogel), recognition of a chemical or biological species such as an enzyme (hydrogel containing immobilized enzymes), an applied magnetic field (magnetic particles dispersed in alginate microspheres), an applied electric field (polyelectrolyte hydrogel), applied UV light (photoresponsive hydrogel), or the application of ultrasound (Ethylene-vinyl alcohol hydrogel).

In one embodiment, the swelling of the gel and concomitant release of the pharmaceutical compound is triggered by a temperature change. The temperature-sensitive hydrogel can exhibit positive thermosensitivity (experience swelling at higher temperature due to the presence of hydrophilic monomers) and negative thermosensitivity (experience swelling at lower temperature due to the presence of hydrophobic monomers). In one embodiment, the temperature-sensitive hydrogel is prepared from a crosslinked poly(N-isopropylacrylamide) which experiences a conformational change above 32° C. (N Peppas et al, J Biomater Sci Polymer Edn, 15, 2, 2004). The swelling temperature can be adjusted by co-polymerization with small amounts of ionic copolymers so that it falls at, slightly below, or slightly above 37° C.

In one embodiment, the swelling of the gel and concomitant release of the pharmaceutical compound is triggered by an increase in the water levels in the epidermis and dermis following completion of the reepithelialization process (removal of the stratum corneum causes loss of water and decreased average water concentrations near the skin's outer surface; when the skin reepithelializes, water levels go back to normal). The water-sensitive hydrogel may be a gel that experiences hydrolysis reactions. In another embodiment, high levels of water cause hydrophobic groups in the hydrogel to aggregate, causing a collapse of the structure thereby releasing the pharmaceutical compound by a “squeezing” process (N Peppas et al, J Biomater Sci Polymer Edn, 15, 2, 2004).

In another embodiment, recognition of a physiological marker differentially expressed during the neogenic window triggers a conformational change in the carrier or causes the cross-linkages of the gel network to break, which in turn causes release of the pharmaceutical compound. For example, high concentrations of matrix metalloproteases caused by abrasion of the skin (Karimipour et al, Journal of the American Academy of Dermatology, 52, Issue 2, 2005) can cause the pharmaceutical compound to be released from the hydrogel matrix in an active form, if the hydrogel is so designed. In the absence of matrix metalloproteases, or in presence of low levels thereof, the pharmaceutical compound is not released, or is released at a much lower rate.

In another embodiment, the carrier is a polyamide microcapsule that can release its pharmaceutical compound contents by photorelease when exposed to UV radiation.

In another embodiment, the particles described in this example can be used in conjunction with the particles described in Example 18. The two types of particles may be supplied in two separate cartridges and applied on the skin at different times or at the same time (e.g. by drawing from both cartridges at the same time and mixing them before they impact the skin), or they can be supplied mixed in one single cartridge and applied on the skin at the same time.

### Example 3

### Composition that Melts at Body Temperature, is Biodegradable, and can be Used in a Conventional Microdermabrasion Device

One or more of the compounds used in the invention (e.g., EGFR inhibitors, retinoids, anti-inflammatories, etc.) are formulated into a low melting fat (e.g., sal fat olein, cocoa butter, palm super olein, and olive oil) or a mixture of low melting and high melting fats (e.g., fully hydrogenated rapeseed oil with a high amount of behenic acid, fully hydrogenated rapeseed oil with a high amount of stearic acid, tristearoyl-glycerol, triarachidonoyl-glycerol, and tribehenoyl-glycerol) by any of several well-established methods, such as disk spinning (Geary et al, Journal of Controlled Release, 23, Issue 1, 1993) or rapid cooling and heating cycles Higaki K et al, Journal of the American Oil Chemists Society, (3), 2003. The fat or mixture of fats has the desirable property of being a solid below body temperature and melting at or near body temperature. The carrier in its solid form (or a mixture of carrier particles and conventional abrasion particles such as alumina) can be propelled against the skin to abrade it. The carrier in its liquid form can dissolve when in contact with a biological tissue, which allows pharmaceutical compound release.

In one embodiment, the fat is second-stage solid fraction (stearin) from palm oil, which melts between 34° C. and 38° C. (Higaki et al, Food Research International, 37 (8), 2004). These particles can be used alone, or in combination with any of the other microdermabrasion particles described herein.

In one embodiment, the fat is a high melting fat which melts at a temperature higher than body temperature but not high enough to damage the skin. One example would be tripalmitin of more than 85% purity, which melts between 61° C. and 65° C.

In this embodiment, the skin is heated to between 61° C. and 65° C. in order to cause release of the pharmaceutical compound.

### Example 4

### Microdermabrasion Devices

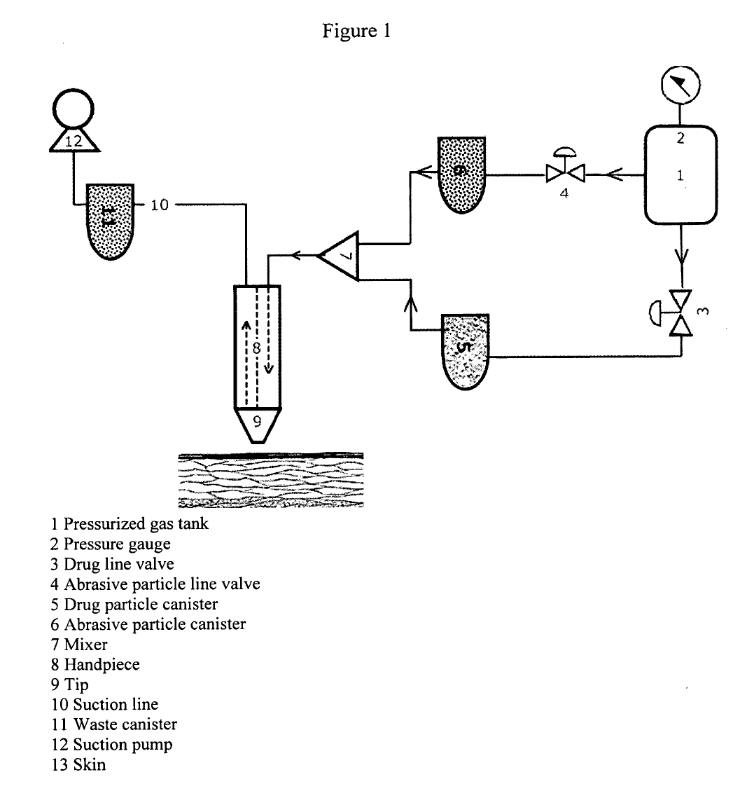

The device in FIG. 1 includes a pressurized gas tank 1 with a pressure gauge 2. Tank 1 supplies pressurized gas through valves 3 and 4. Valve 3 regulates the pressure of the gas entering drug cartridge 5, and can therefore be used to control the speed of the drug particles entrained by the gas. Valve 4 regulates the pressure of the gas entering abrasive particle cartridge 6, and can therefore be used to control the speed of the abrasive particles entrained by the gas. The relative aperture of valves 3,4 determines whether the gas flow entrains abrasive particles only, drug particles only, or a mixture of both, as well as the relative proportions of a mixture. By way of example but not by way of limitation, drug particle cartridge 5 may contain particles made of a polymer carrier that can release a drug in a controlled manner. By way of example but not by way of limitation, abrasive particle cartridge 6 may contain abrasives such as alumina particles or other metal oxides, sodium chloride, or sodium bicarbonate. Alternatively (not shown), the device can include on single cartridge which contains a mixture of abrasive and drug particles. The gas with entrained particles from cartridge 5, 6, or both, enters mixer 7, where the flow simply passes by if only one of the lines is in use, or the flows from cartridges 5, 6 mix forming a homogeneus gas mixture, if both lines are in use. The gas with entrained particles flows from mixer 7 to handpiece 8, which directs it, through tip 9, against skin 13. The handpiece 8 is contacted with the skin of a patient by moving it horizontally on the surface of the skin. The handpiece also has a waste recovery line 10, through which skin debris fragments and leftover particles are removed from the surface of the skin. This conduit is coupled to a suction pump 12 which regulates the level of the vacuum. The suction directs the waste to a waste canister 11. A mode of use may consist of first opening valve 4 while keeping valve 3 closed, so that gas entrains only abrasive particles but not drug particles. On a first pass, abrasive particles from cartridge 6 are used to abrade the superficial layers of the skin. The abrasive power of the gas stream is determined by the size and hardness of the abrasive particles and the aperture of valve 4, which regulates the pressure and therefore the velocity of the gas stream. The aperture of valve 4 is chosen so that abrasive particles have enough momentum to remove the superficial layers of the skin but not enough momentum to penetrate them and embed deep into the skin. In order to ensure thorough removal of the waste generated (skin debris and leftover abrasive particles), the suction in line 10 is kept at a high level by suction pump 12. When this first step is completed, valve 4 is closed and the level of vacuum in suction pump 12 is reduced or eliminated altogether by shutting down the pump. On a second pass, valve 3 is opened and the gas stream entrains drug particles from cartridge 7. These particles are meant to be propelled against the skin so that they penetrate it and embed in it. The penetration depth of the particles is determined by the properties of the skin and by the particle size, shape, velocity, and density. The practitioner implementing the treatment will know in advance the properties of the skin and the characteristics of the particles, and will use aperture of valve 3 as a means for regulating the particle velocity and penetration depth. During this step, little or no vacuum is applied by suction pump 12 in order to minimize the losses of drug particles due to suction.

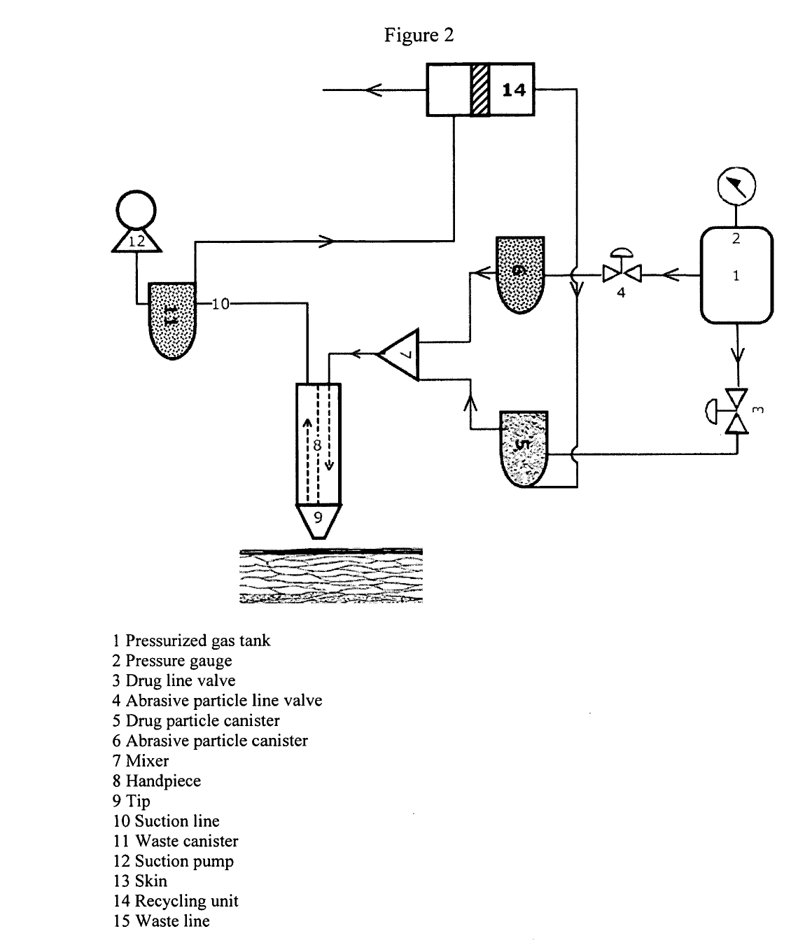

The device set forth in FIG. 2 includes a recycling unit 14 downstream of cartridge 11. In recycling unit 14 drug particles are separated from skin debris and, optionally from other abrasive particles. By way of example but not by way of limitation, one separation method can consist of a sieve that allows small drug particles to cross through but retains larger abrasive particles or skin fragments. The fraction of waste retained exits the recycling unit through waste line 15, and is subsequently discarded, or recirculated back to waste cartridge 11 for further rounds of purification (not shown). The drug particles that cross the sieve are recovered and recirculated to drug cartridge 5 for re-use.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1-104. (canceled)

105. A method of enhancing hair growth in a subject, said method comprising:
(i) disrupting the dermis or epidermis of said subject; and (ii) embedding particles into said dermis or epidermis, wherein at least some of said particles comprise a pharmaceutical compound that is released into said dermis or epidermis, and wherein said embedding comprises propelling said particles into said dermis or epidermis.

106. The method of claim 105, wherein said disrupting comprises a light-based method.

107. The method of claim 106, wherein said light-based method employs a laser.

108. The method of claim 107, wherein said laser is a fractional laser, CO₂ laser, or excimer laser.

109. The method of claim 105, wherein said disrupting comprises perforating said dermis or epidermis.

110. The method of claim 105, wherein said disrupting comprises abrading said dermis or epidermis.

111. The method of claim 110, wherein said abrading comprises microdermabrasion.

112. The method of claim 111, wherein said microdermabrasion comprises propelling said particles comprising said pharmaceutical compound.

113. The method of claim 105, wherein said embedding comprises setting said particles securely within the surface of the dermis or epidermis.

114. The method of claim 105, wherein said particles stick to the dermis or epidermis.

115. The method of claim 105, wherein said particles are between 0.01 μm and 200 μm in diameter.

116. The method of claim 115, wherein said particles are between 1 μm to 100 μm in diameter.

117. The method of claim 105, wherein said particles are formulated for controlled release of said pharmaceutical compound.

118. The method of claim 117, wherein said release is activated by an exogenous or endogenous stimulus.

119. The method of claim 105, wherein said particles are biocompatible.

120. The method of claim 105, wherein said method further comprises eliminating or collecting said particles following release of said pharmaceutical compound.

121. A method of treating a scarred region in a subject, said method comprising: (i) disrupting the dermis or epidermis of said subject; and (ii) embedding particles into said dermis or epidermis, wherein at least some of said particles comprise a pharmaceutical compound that is released into said dermis or epidermis, and wherein said embedding comprises propelling said particles into said dermis or epidermis.

122. The method of claim 121, wherein said disrupting comprises a light-based method.

123. The method of claim 122, wherein said light-based method employs a laser.

124. The method of claim 123, wherein said laser is a fractional laser, CO₂ laser, or excimer laser.

125. The method of claim 121, wherein said disrupting comprises perforating said dermis or epidermis.

126. The method of claim 121, wherein said disrupting comprises abrading said dermis or epidermis.

127. The method of claim 126, wherein said abrading comprises microdermabrasion.

128. The method of claim 127, wherein said microdermabrasion comprises propelling said particles comprising said pharmaceutical compound.

129. The method of claim 121, wherein said embedding comprises setting said particles securely within the surface of the dermis or epidermis.

130. The method of claim 121, wherein said particles stick to the dermis or epidermis.

131. The method of claim 121, wherein said particles are between 0.01 μm and 200 μm in diameter.

132. The method of claim 131, wherein said particles are between 1 μm to 100 μm in diameter.

133. The method of claim 121, wherein said particles are formulated for controlled release of said pharmaceutical compound.

134. The method of claim 133, wherein said release is activated by an exogenous or endogenous stimulus.

135. The method of claim 121, wherein said particles are biocompatible.

136. The method of claim 121, wherein said method further comprises eliminating or collecting said particles following release of said pharmaceutical compound.
